(19) United States
(12) Patent Application Publication
(10) Pub. No.: US 2020/0155999 A1
(43) Pub. Date: May 21, 2020

(54) An Altitude Simulation Assembly

(71) Applicant: SPORTING EDGE (UK) LIMITED, Hampshire (GB)

(72) Inventor: David Vincent, Sherfield on Loddon, Hants (GB)

(21) Appl. No.: 16/627,493

(22) PCT Filed: Jul. 5, 2018
(86) PCT No.: PCT/GB2018/000104

§ 371 Date: Dec. 30, 2019

(30) Foreign Application Priority Data
Jul. 5, 2017 (GB) 1710778.0

(51) Int. Cl. B01D 53/22; A61M 16/16; B01D 53/30; A61M 16/00; B01D 53/26; A61M 16/10

(52) U.S. Cl. CPC B01D 53/22; A61M 16/16; B01D 53/30; A61M 16/0063; B01D 53/265; A61M 16/101; A61M 2205/7536; A61M 16/1065; A61M 16/022; A61M 2016/1025; B01D 2053/224; A61M 2205/3606; B01D 53/266

(57) ABSTRACT

An altitude simulation assembly for an environmental chamber includes: at least one ambient air inlet; an air compressor downstream of said ambient air inlet for compressing the ambient air; at least one gas separation means downstream of the air compressor for separating the compressed air into hypoxic gas and hyperoxic gas; and, at least one fluid flow control means in fluid communication with the at least one gas separation means, for controlling the flow of hypoxic gas and hyperoxic gas to the environmental chamber. The at least one fluid flow control means is in fluid communication with at least one outlet port for supplying hypoxic gas from the gas separation means to the at least one outlet port, and hyperoxic gas from the gas separation means to the at least one outlet port. The fluid flow control means controls the oxygen concentration of gas to the environmental chamber.

### CROSS-REFERENCE TO RELATED APPLICATIONS

This is the U.S. National Stage application of International Application No. PCT/GB2018/000104, filed Jul. 5, 2018, which claims the benefit of priority from GB Application No. 1710778.0, filed Jul. 5, 2017. The entire contents of these prior applications are incorporated by reference herein.

### FIELD

This invention pertains generally to the field of altitude simulation, and in particular altitude simulation through variation of oxygen concentration levels within a room, exercise space or environmental chamber or surrounding environment.

### BACKGROUND

Hypoxic generators are used during hypoxic therapy by individuals to obtain the benefits in physical performance and wellbeing through improved oxygen metabolism. A hypoxic generator is a device that is used to deprive the body of an adequate oxygen supply. These generators comprise apparatus to provide reduced oxygen, or hypoxic air to a user for active or passive simulated altitude training Hypoxic gas typically contains less than 21% oxygen concentration.

Incorporating some element of exposure to reduced oxygen atmospheres into a training program can be beneficial in terms of performance and general well-being. It has become a widely used element of training for elite athletes and is starting to become used at lower levels as well as for pre-acclimatisation before travelling to high altitude climates and for maintaining fitness levels when suffering from injury. Typically, the existing systems reduce the concentration of oxygen within an enclosed environment, which therefore has the effect of increasing the simulated altitude within that environment. In many of these devices, compressed air is passed through a molecular sieve to separate the air into a hypoxic air stream (reduced oxygen) and a hyperoxic air stream (increased oxygen). The hypoxic air stream is conventionally used to create simulated high altitude conditions for training and therapy purposes within a particular air space or environmental chamber.

In such systems, a user would set the simulated altitude to a specific level, and the control system within the device would maintain this level by supplying hypoxic air to the air space. The hyperoxic air stream is typically discarded.

However, where a person resides at elevated altitude, or visits such a location for an extended period of time, the person is permanently exposed to a reduced oxygen environment. Respiring within such an environment on a permanent basis can have a detrimental effect on their musculoskeletal system, likely causing their muscles to reduce in size, even though their cardiovascular system would likely become more efficient at extracting oxygen from such thinner air. A person experiencing such an exposure to high altitude and therefore low oxygen air, would benefit from frequent exposure to simulated sea level, or similar low altitude, conditions. A simulated lower altitude environment, with therefore an increased oxygen content of the surrounding air, would help a person to retain muscle mass, and be provided with improved, and likely faster, recovery from injury or ailments.

The prior art shows a number of devices which attempt to address these needs in various ways.

US 2007 221 225 (Kutt et al) discloses a means of controlling hypoxic air and normoxic air from a hypoxic/normoxic air unit to modulate hypoxia and to control air quality supplied to a user when simulating an atmospheric environment corresponding to a desired altitude to thereby initiate a physiological response. In particular, a method and apparatus for simulating altitude within an enclosure are disclosed. The apparatus comprises an hypoxic air generator configured to provide hypoxic air to an enclosure; a re-circulated air intake for providing air from the enclosure to the hypoxic air generator; a fresh air intake, an air shut-off that is movable between a first position for allowing air from the fresh air intake to enter the chamber, thus preventing air from the re-circulated air intake to enter the chamber, and a second position for allowing air from the re-circulated air intake to enter the chamber, and preventing air from the fresh air intake to enter the chamber. The air shut-off is controlled by an actuator, wherein the actuator receives control information from a controller for controlling the oxygen content of the enclosure. The apparatus is configured to supply either hypoxic air stream or a fresh air stream to the chamber in response to set requirements on the control means.

Whilst the prior art appears to address the issue of having the capability to decrease the oxygen concentration levels within an environment, and therefore simulating high altitude conditions within the environment, this prior art does not provide means to supply hyperoxic gas to the environment to allow an increase in oxygen concentrations, and to be able to take the level of oxygen to above that existing in ambient air at the location. The prior art attempts to address the issue of reducing the simulated altitude within an environment, however it does so by supplying ambient air surrounding the room to the chamber, and therefore it does not provide a solution to being able to reduce this simulated altitude to below the surrounding environmental conditions of the location. The prior art found comprise systems and apparatus that incorporate a considerable number of valves, which is likely to increase the risk of failure and therefore decrease the reliability of the system.

### BRIEF SUMMARY

Preferred embodiments of the present invention aim to provide an altitude simulation assembly with improved control of oxygen concentration within a specific environment, that allows the environment to provide simulated increased altitude and simulated reduced altitude conditions, as and when required.

According to one aspect of the present invention, there is provided an altitude simulation assembly for an environmental chamber, the altitude simulation assembly comprising:
at least one ambient air inlet;
an air compressor downstream of said ambient air inlet for compressing the ambient air;
at least one gas separation means downstream of the air compressor for separating the compressed air into hypoxic gas and hyperoxic gas; and,
at least one fluid flow control means in fluid communication with the at least one gas separation means, for controlling the flow of hypoxic gas and hyperoxic gas to the environmental chamber,
   whereby said at least one fluid flow control means is in fluid communication with at least one outlet port for supplying hypoxic gas from the gas separation means to the at least one outlet port, and hyperoxic gas from the gas separation means to the at least one outlet port, whereby, in use, said fluid flow control means controls the oxygen concentration of gas to the environmental chamber.

Preferably, the fluid flow control means comprises at least one electrically activated valve.

Preferably, the electrically activated valve is operatively connected to a first outlet port for supplying hypoxic gas to the environmental chamber, and a second outlet port for supplying hyperoxic gas to the environmental chamber.

The electrically activated valve may be a solenoid valve configured to move between a first position and a second position, whereby in a first position the solenoid valve is configured to supply hypoxic gas to the environmental chamber and in a second position the solenoid valve is configured to supply hyperoxic gas to the environmental chamber.

Preferably, the air compressor is in fluid communication with an air cooling means for condensing water vapour in the compressed air.

The air cooling means may comprise a fan-cooled radiator.

Preferably, the air cooling means is in fluid communication with a water removal filter for removing condensed water vapour from the compressed air.

The water removal filter may incorporate an automatic drain for releasing condensed water vapour that has been retained in the filter.

The at least one gas separation means may comprise at least one hollow fibre membrane.

In fluid communication with the at least one outlet port may be at least one respiratory filter for removing contaminants from the hypoxic and/or hyperoxic gas.

The at least one outlet port may be in fluid communication with a fluid delivery pipe.

Preferably, the fluid flow control means is controlled by a control device, said control device comprising a control panel and display.

Preferably, the control means is operatively connected to at least one oxygen sensor, whereby said at least one oxygen sensor is configured to sense oxygen level at a location within the environmental chamber.

The fluid flow control means is configured to supply a variable concentration of oxygen to the environmental chamber to alter the simulated altitude within said environmental chamber.

The aforementioned altitude simulation assembly may be configured such that the assembly is retrofittable to an existing gas delivery system.

### BRIEF DESCRIPTION OF THE DRAWINGS

For a better understanding of the invention and to show how embodiments of the same may be carried into effect, reference will now be made, by way of example, to the accompanying diagrammatic drawings, in which:

FIG. 1 shows a flow diagram of one embodiment of an altitude simulation assembly for delivering hypoxic gas and/or hyperoxic gas to an environmental chamber;

FIG. 2 shows the flow diagram of FIG. 1, with one embodiment of gas separation means in fluid communication with at least one electrically activated valve, configured to deliver hypoxic gas and hyperoxic gas to the environmental chamber through separate outlet ports;

FIG. 3 shows a further embodiment of gas separation means in fluid communication with at least one electrically activated valve, configured to deliver hypoxic gas and hyperoxic gas to the environmental chamber through the same outlet port; and,

FIG. 4 shows a further embodiment of fluid flow control means, where the at least one electrically activated valves comprise a solenoid valve.

In the figures like references denote like or corresponding parts.

### DESCRIPTION OF EXAMPLE EMBODIMENTS

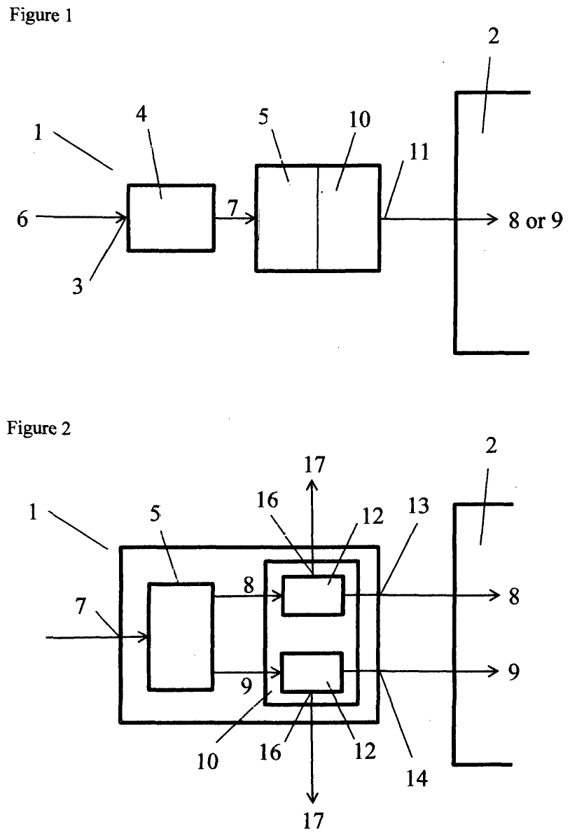

As shown in FIG. 1, an altitude simulation assembly 1 comprises an air compressor 4 that draws in ambient air 6 through an ambient air inlet 3, compresses this ambient air stream 6 to form compressed air 7 and provides this compressed air 7 to a gas separation means 5. The gas separation means 5 separates the compressed air 7 into hypoxic gas 8 and hyperoxic gas 9, which are passed to a flow control device that provides one or other to the chamber according to that required within an environmental chamber 2. The gas separation means 5 delivers the hypoxic gas 8 and/or hyperoxic gas 9 to the environmental chamber 2 through at least one outlet port 11.

The altitude simulation assembly 1 supplies air to the environmental chamber 2 with a specific concentration of oxygen. This alters the overall oxygen level within the environmental chamber 2 to simulate a different altitude within the environmental chamber 2. Reducing the concentration of oxygen of the air within the environmental chamber 2, raises the simulated altitude within the environmental chamber 2. Increasing the concentration of oxygen of the air within the environmental chamber 2, lowers the simulated altitude within the environmental chamber 2. The gas separation means 5 is controlled to allow for this change in simulated altitude.

FIG. 2 shows one embodiment of gas separation means 5 when in fluid communication with at least one electrically activated valve 12. Shown in this figure are a pair of electrically activated valves 12. These electrically activated valves 12 operate as the fluid flow control means 10 and are configured to control the hypoxic gas 8 and the hyperoxic gas 9 being emitted by the gas separation means 5. In this embodiment each electrically activated valve is configured to control either the flow of hypoxic gas 8 or hyperoxic gas 9 to the environmental chamber 2. Each electrically activated valve 12 is in fluid communication with a vent 16 to vent any waste gas to the atmosphere 17 external to the environmental chamber 2.

The hypoxic gas 8 and hyperoxic gas 9 are supplied to the environmental chamber 2 through two separate outlet ports 11, a first outlet port 13 configured to supply hypoxic gas 8 to the environmental chamber 2 and a second outlet port 14 configured to supply hyperoxic gas 9 to the environmental chamber 2.

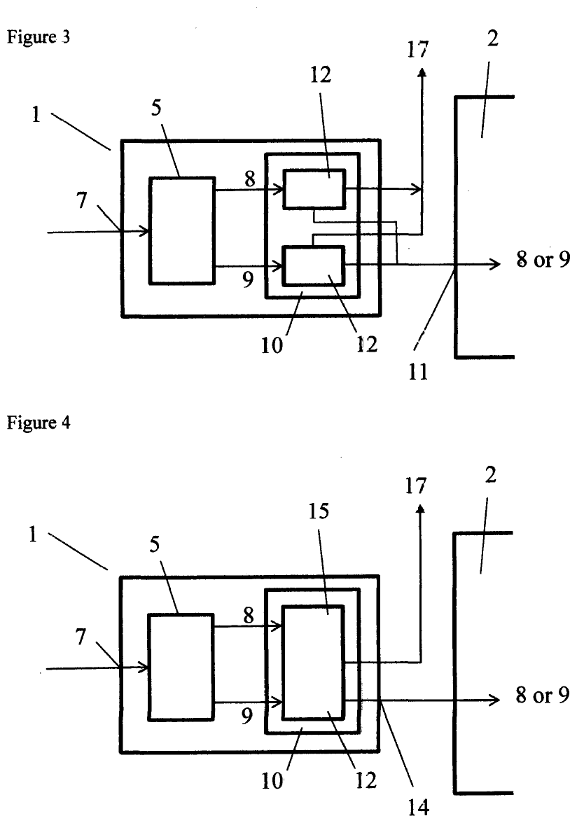

FIG. 3 shows a further arrangement of gas separation means 5 fluidly connected to a pair of electrically activated valves 12, configured to control the concentration of oxygen within the air flow that is supplied to the environmental chamber 2, whereby only one outlet port 11 supplies either hypoxic gas 8 or hyperoxic gas 9 to the environmental chamber 2. The first outlet port 13 is configured to supply waste gas through a vent 16 and to the atmosphere 17. The electrically activated valves 12 are operated so that either hypoxic gas 8 or hyperoxic gas 9 are passed to the environmental chamber 2 via the port 11 and the gas other than that being supplied to the environmental chamber 2 is passed to the atmosphere 17.

FIG. 4 shows a further arrangement where the fluid flow control means may comprise a single unit with at least one solenoid valve 15 operating as the electrically activated valves 12. It may also comprise a rotary valve that is motor driven, not shown. The solenoid valve 15 may also be replaced by other activated valves, that include, but are not restricted to, pneumatic valves.

Entry into the environmental chamber may be through a single, dual, plurality of arrangement of fluid delivery pipes, or similar ducting that allows for sufficient environmental chamber 2 distribution. The altitude simulation assembly 1 allows a considerable variance of altitude to be simulated within the environmental chamber 2, ranging from sea level to the altitude experienced at the top of the highest peak of a mountain range such as Mount Everest.

The system will likely be controlled by a control device, with control panel provided with a plurality of controls or buttons, a display, and in electrical communication with at least one oxygen sensor. There may be a plurality of oxygen sensors placed throughout the environmental chamber 2 to enable a reading of the overall oxygen concentration of air within the environmental chamber 2 to be determined. The control device may be programmed to interpret the data and control the fluid flow control means accordingly.

1-15. (canceled)

16. An altitude simulation assembly for an environmental chamber, the altitude simulation assembly comprising:
at least one ambient air inlet;
an air compressor downstream of said at least one ambient air inlet for compressing ambient air into compressed air;
at least one gas separation means downstream of the air compressor for separating the compressed air into hypoxic gas and hyperoxic gas; and,
a fluid flow control means in fluid communication with the at least one gas separation means, for controlling the flow of hypoxic gas and hyperoxic gas to the environmental chamber,
the fluid flow control means being in fluid communication with at least one outlet port for supplying hypoxic gas from the at least one gas separation means to the at least one outlet port and hyperoxic gas from the at least one gas separation means to the at least one outlet port, said fluid flow control means controlling the oxygen concentration of gas to the environmental chamber.

17. An altitude simulation assembly according to claim 16, wherein the fluid flow control means comprises at least one electrically activated valve.

18. An altitude simulation assembly according to claim 17, wherein the at least one outlet port includes a first outlet port and a second outlet port and the electrically activated valve is operatively connected the first outlet port for supplying hypoxic gas to the environmental chamber and the second outlet port for supplying hyperoxic gas to the environmental chamber.

19. An altitude simulation assembly according to claim 17, wherein the electrically activated valve is a solenoid valve configured to move between a first position and a second position, whereby in a first position the solenoid valve is configured to supply hypoxic gas to the environmental chamber and in a second position the solenoid valve is configured to supply hyperoxic gas to the environmental chamber.

20. An altitude simulation assembly according to claim 16, wherein the air compressor is in fluid communication with an air cooling means for condensing water vapor in the compressed air.

21. An altitude simulation assembly according to claim 20, wherein the air cooling means comprises a fan-cooled radiator.

22. An altitude simulation assembly according to claim 20, wherein the air cooling means is in fluid communication with a water removal filter for removing condensed water vapor from the compressed air.

23. An altitude simulation assembly according to claim 22, wherein the water removal filter incorporates an automatic drain for releasing condensed water vapor that has been retained in the filter.

24. An altitude simulation assembly according to claim 16, wherein the at least one gas separation means comprises at least one hollow fiber membrane.

25. An altitude simulation assembly according to claim 16, wherein in fluid communication with the at least one outlet port is at least one respiratory filter for removing contaminants from the hypoxic and/or hyperoxic gas.

26. An altitude simulation assembly according to claim 16, wherein the at least one outlet port is in fluid communication with a fluid delivery pipe.

27. An altitude simulation assembly according to claim 16, wherein the fluid flow control means is controlled by a control device, said control device comprising a control panel and display.

28. An altitude simulation assembly according to claim 27, wherein the fluid flow control means is operatively connected to at least one oxygen sensor, the at least one oxygen sensor being configured to sense an oxygen level at a location within the environmental chamber.

29. An altitude simulation assembly according to claim 16, wherein the fluid flow control means is configured to supply a variable concentration of oxygen to the environmental chamber to alter a simulated altitude within said environmental chamber.
